(19) United States
(12) Patent Application Publication
(10) Pub. No.: US 2023/0037979 A1
(43) Pub. Date: Feb. 9, 2023

(54) Double Lumen Pigtail Catheter and HOCM Gradient Catheter

(71) Applicants: Wesley Robert Pedersen, Minneapolis, MN (US); William Joseph Drasler, Minnetonka, MN (US)

(72) Inventors: Wesley Robert Pedersen, Minneapolis, MN (US); William Joseph Drasler, Minnetonka, MN (US)

(21) Appl. No.: 17/817,668

(22) Filed: Aug. 5, 2022

Related U.S. Application Data
(60) Provisional application No. 63/229,693, filed on Aug. 5, 2021.

(51) Int. Cl. A61M 25/00; A61M 25/01

(52) U.S. Cl. CPC A61M 25/0021; A61M 25/01; A61M 2210/125

(57) ABSTRACT

Pigtail catheters and relates methods for measuring a pressure gradient across a bodily narrowing are disclosed. A pigtail catheter can comprise a proximal shaft segment and a distal shaft segment. The proximal shaft segment can include double lumen tubing defining a proximal pressure lumen and a non-coaxial, distal pressure lumen. In an example, the distal pressure lumen has a generally circular cross-sectional shape, and the proximal pressure lumen has a generally crescent or kidney cross-sectional shape that wraps partially around the distal pressure lumen. The distal shaft segment can include at least one distal orifice positionable distal to the bodily narrowing and at least one proximal orifice positionable proximal to the bodily narrowing. Each orifice can have a diameter of at least about 0.018 inches, for example. A manifold can be coupled to a proximal end of the proximal shaft segment and can include a proximal pressure port in communication with the proximal pressure lumen and a distal pressure port in communication with the distal pressure lumen.

### CLAIM OF PRIORITY

This non-provisional patent document claims the benefit of priority under 35 U.S.C. § 119(e) to Pedersen et al., U.S. Provisional Patent Application Ser. No. 63/229,693, entitled “DOUBLE LUMEN PIGTAIL CATHETER AND HOCM GRADIENT CATHETER” and filed on Aug. 5, 2021, which is herein incorporated by reference in its entirety.

### TECHNICAL FIELD

This patent document relates to medical devices. More particularly, but not by way of limitation, the patent document relates to catheters.

### OVERVIEW

Dual (or double) lumen pigtail catheters can be used in interventional procedures to measure a pressure gradient across native valves or bioprosthetic valves of the heart, across stenoses within native vascular lumens of the body, or across other narrowings found within non-vascular cavities or tubular members of the body. A distal region of a pigtail catheter that is adapted to measure a distal pressure can be placed distal to a stenosis or narrowing, and a proximal region of the pigtail catheter that is adapted to measure a proximal pressure can be placed proximal to the stenosis or narrowing to allow measurement of a pressure gradient across the stenosis or narrowing. Measurement of a pressure gradient across an aortic valve, for example, can be accomplished by placing a distal region of a pigtail catheter into the left ventricle (LV) to measure the LV pressure and positioning a more proximal region of the pigtail catheter in the ascending aorta to measure the aortic pressure. A high-pressure gradient across the aortic valve is indicative of aortic valve stenosis, which can be treated via valvuloplasty, transcatheter aortic valve replacement, surgical valve replacement, medications, or other treatment methods. Other stenoses can be treated via placement of a stent, for example, to enlarge a stenosis found in a native arterial or prosthetic conduit or other non-vascular organ conduit of the body.

Dual lumen pigtail catheters in current use are formed with a catheter shaft including coaxial lumens and are associated with drawbacks. Such catheters are susceptible to recording less reliable pressure signals from the ascending aorta or left ventricle, for example, to a pressure transducer that is connected to a proximal end of the pigtail catheter. Often the pigtail portion or adjacent distal region of the catheter shaft can be susceptible to kinking which, in combination with a smaller transmission lumen, can result in attenuated signal transmission leading to unreliable pressure gradients. Torqueability of the catheter shaft for distal positioning is also lacking.

The present inventors recognize that what is needed is a low-profile double lumen pigtail catheter that will accurately transmit pressure signals from a region both proximal and distal to a stenosis or narrowing to a proximal end of the catheter. When using such a pigtail catheter for measurement of a pressure gradient across a stenotic aortic valve, for example, the pigtail catheter should be able to transmit a highly accurate, non-attenuated, and frequency responsive pressure signal simultaneously from the LV and the ascending aorta to the proximal end of the catheter. The pigtail portion of the catheter should be resistant to kinking, have excellent torque transmission to the distal catheter shaft, have a biased distal catheter shaft bend, and/or optimal LV pigtail signal transmission. In some instances, the pigtail catheter should be able to be delivered to the LV over a low-profile diagnostic cardiology catheter, for example, that can be used to provide safer and less traumatic access across a stenotic aortic valve.

Measuring a pressure gradient across a stenotic intra LV cavity segment, such as hypertrophic obstructive cardiomyopathy (HOCM), can be difficult to accurately localize and measure proximal and distal pressures. A hypertrophic region of cardiac muscle can protrude from the proximal septal wall of the LV, for example, and extend into the left ventricular outflow tract (LVOT) adjacent to the anterior mitral valve leaflet. This protrusion can cause blood flow through the LVOT to have a higher velocity than normal, thereby producing a localized low-pressure region that can pull the anterior mitral leaflet toward the protrusion resulting in an even greater restriction to blood flow than that cause by the protrusion alone. The result is a dynamic pressure drop across the LVOT narrowing.

Typically, the hypertrophic segment of cardiac muscle can be treated surgically or ablated by transcatheter alcohol ablation. Intra LV pressures need to be discretely localized in segment proximal and distal to the dynamic obstruction. This is often made difficult by the short distance between the aortic valve and proximal segment of the septal hypertrophy, which need to be localized for proximal pressure measurement. Localizing the pressure to proximal to the LVOT gradient is especially relevant in patients that have combined HOCM and aortic stenosis. In the clinical scenario where there are two distinct systolic gradients, they need to be quantified separately to determine appropriate treatment strategies, such as HOCM septal ablation, TAVR, or both. In addition, patients with HOCM frequently have hyperdynamic LV systolic function that can result in distal cavity-end systolic collapse which can impinge on the distal orifice and impair accurate pressure measurement.

The present inventors recognize that what is needed is a gradient catheter having a coil with distal orifices or a distal opening located distal to the obstruction; the coil can have a small diameter and be shaped to prevent impingement of the distal orifices or distal opening. Proximal orifices located proximal to the obstruction should be accurately positioned in the proximal LVOT and distal to the aortic valve. Proximal and distal pressures can be measured simultaneously to quantitate the dynamic stenosis. The baseline gradient will determine the severity of the obstruction. The pressure gradient can be measured from a location within the LV to a location just below or adjacent to the aortic valve within the LVOT. The catheter should be able to place the proximal orifice proximal to the stenotic aortic leaflets within the aorta while maintaining the coil proximal to the dynamic obstructive segment in the LVOT without orifice blockage, thereby providing an ability to measure a separate end-systolic gradient across a stenotic aortic valve.

### SUMMARY

The present invention includes a pigtail catheter formed from a double lumen extrusion, for example, that does not have coaxial lumens. The double lumen tubing can provide two lumens having separate axes that run parallel to each other but are not coaxial. It is believed that this double lumen arrangement can provide a greater hydraulic diameter for each of the two lumens in comparison to a dual lumen catheter having coaxial lumens, thereby providing improved pressure signal transmission from a distal shaft segment to a manifold located at a proximal end of the pigtail catheter.

A proximal shaft segment, which includes the double lumen shaft tubing, can be braided to provide torque transmission characteristics to the distal shaft segment of the catheter. The catheter shaft can have a shaft bend in the axial direction of the LV thereby allowing the catheter to extend into the LV without impinging upon the LV inferior and septal segments, most notably, which could result in arrhythmic abnormalities that would render unreliable assessment of both pressure gradient and left ventricular systolic function. The distal shaft segment of the catheter adjacent to the pigtail coil can be supported by either braiding or an elastic member that resists kinking and provides torque transmission to the distal shaft segment, while preserving flexibility to the coil preventing trauma to submitral valve structures and having less resistance to wire exchanges.

The pigtail coil of a catheter embodiment can form a coil plane that is coplanar with a bend plane formed by the catheter shaft on the proximal and distal sides of the shaft bend; the coil plane can be directed toward the anterolateral left ventricular chamber by applying counterclockwise torque to the proximal portion of the catheter to optimize contrast opacification of the LV. In another embodiment, the pigtail catheter can form a coil plane that is not coplanar with the bend plane. The coil plane can be angled relative to the shaft bend plane to position the pigtail. In an embodiment, the distal pressure lumen can be sized and shaped to have a diameter that will allow passage of a low-profile diagnostic cardiology catheter that may be used to assist in delivery of a straight-tipped or other guidewire followed by a diagnostic catheter from the aorta to the LV across a stenotic aortic valve. Such diagnostic cardiology catheters may include Amplatz Left (AL), multipurpose catheters, right Judkins catheters, and other catheter configurations and will hereinafter be referred to as cardiac diagnostic catheters. Advancement of the pigtail catheter over the cardiac diagnostic catheter after first pulling the straight tipped crossing wire back into the diagnostic catheter can provide a less traumatic method, whereby the pigtail catheter uses the diagnostic catheter as a rail to deliver the pigtail catheter without a straight-tip guidewire freely exposed to the LV apex.

The pigtail catheter can undergo some specific modifications to provide a catheter to accurately measure a dynamic obstruction within the LV chamber present in hypertrophic obstructive cardiomyopathy (HOCM) in the LVOT secondary to a hypertrophic segment in the proximal LV septum. The HOCM pigtail catheter may have any of the features that are described for any embodiment of the cardiac pigtail catheter, including a braided shaft for various shaft regions to enhance torqueability and provide anti-kinking character, a double lumen shaft to enhance pressure signal transmission via two lumens of adequate hydraulic diameter within the double lumen shaft, a flexible coil located at the distal end of the pigtail catheter, orifices located along portions of the shaft or in the coil, a distal opening in the coil that has adequate diameter to allow passage over a guidewire and provide an opening for pressure signal transmission from the body chamber to the shaft lumen, orifice diameter that allow pressure signal transmission from the body chamber to the shaft lumen, and/or a shaft bend angle that positions the coil within the LV without eliciting ectopic signals from the myocardium which renders dynamic LVOT obstructions spurious.

The HOCM pigtail catheter of the present invention can have orifices and a distal opening that are protected from myocardial tissue impingement onto the orifices or distal opening during systolic contraction of the heart. Such systolic contraction can cause myocardial tissue to partially block the orifice or distal opening resulting in attenuated pressure signal transmission from the heart chamber to the shaft lumen resulting in an error in the pressure gradient measurement. Protection to the orifices can be obtained by placing the orifices on the innermost edge of the circular or oval shaped coil. The orifices can have an oval shape to enhance the area of the opening and make them harder for myocardial tissue to block signal transmission. The distal opening at the end of the coil can be protected by placing the distal opening near or abutting the proximally adjacent catheter shaft that can assist in holding myocardial tissue away from the distal opening. Since the distance from the hypertrophic cardiac muscle to the aortic valve can be short, about 5 mm (range of 2-10 mm), the proximal orifice region can contain only about one or two orifices such that the proximal orifice region can be accurately placed below the aortic annulus and proximal to the hypertrophic septal segment. A radiopaque marker can be located about 1 mm proximal to the proximal orifice region, for example, to allow the operator to visualize the location of the proximal orifice region under fluoroscopy.

A coil diameter for the HOCM pigtail catheter can be smaller in diameter with a diameter of 5 mm (range 3-10 mm, for example). The smaller coil diameter can better localize the pressure distal of the LVOT obstruction during the hyper dynamic systolic contraction generally seen in HOCM patients in the more distal LV chamber. To better avoid occlusion of the distal orifice by the myocardial tissue during systole, the post-bend region of the pigtail catheter can have a length of about 4 cm, for example, to maintain the coil at about 2-3 cm from the apex of the LV. A radiopaque marker located at the coil distal opening can aid in the accurate fixing of the catheter segment for measuring the distal pressure distal to the obstructive segment. Additionally, a radiopaque marker can be located near the proximal orifice that is positioned adjacent to the aortic valve in the LVOT.

The double lumen shaft of the present invention can alternately be formed without a braid contained within its outer wall. In an alternate embodiment, a fiber or ribbon can be placed into the outer shaft wall during the extrusion process or via other processing methods at a location close to the center of the oval proximal pressure lumen, for example. The presence of such a fiber or ribbon can enable and direct any bending incurred by the double lumen shaft such that the minor diameter of the oval proximal pressure lumen is not reduced. Thus, the fidelity of the pressure signal delivered by the proximal pressure lumen can be retained when the catheter shaft is being bent.

In another embodiment, the outer surface of the double lumen shaft can be formed with an oval shape. The outer major axis can be directed along a line extending through a center of the distal pressure lumen and through the center of the proximal pressure lumen. Such an oval outer surface shape can provide the proximal pressure lumen of the double lumen shaft to have a greater minor diameter than can be attained with a round double lumen shaft of the same perimeter and thereby maintain the perimeter of the outer surface of the double lumen shaft at a minimum. The oval double lumen shaft can pass through a smaller introducer catheter than a round double lumen shaft having the same minor diameter for the proximal pressure lumen. The result can be a greater pressure signal fidelity while passing the double lumen shaft through a smaller profile introducer sheath.

Several factors can affect the fidelity of the pressure signal that is transmitted back to the pressure transducer located at or near the manifold. Fluid resistance created by the viscosity of a fluid moving through a small diameter proximal pressure lumen, for example, can reduce the magnitude of the pressure signal that is transmitted to the pressure transducer. The hydraulic diameter of the proximal pressure lumen should therefore be maintained at a dimension of at least about 0.018 inches to ensure a fidelity signal transmission capability. A long tubing length can affect the inertia of the fluid moving through the catheter shaft and result in a phase delay of the pressure signal that is being transmitted back to the pressure transducer. Tubing compliance can cause the pressure signal to become attenuated, and phase delayed in reaching the pressure transducer. Such variables can be examined mathematically and chosen to optimize the fidelity of the signal that is transmitted from the proximal orifices to the proximal pressure lumen of the present double lumen catheter to the pressure transducer located at the manifold.

These and other examples, features and findings of the present catheters and related methods will be set forth, at least in part, in the following Detailed Description. This Summary is intended to provide non-limiting examples of the present teachings—it is not intended to provide an exclusive or exhaustive explanation. The Detailed Description below is included to provide further information about the present catheters and related methods.

### BRIEF DESCRIPTION OF DRAWINGS

The drawings illustrate generally, by way of example, but not by way of limitation, various embodiments discussed in this patent document.

FIG. 1A is a plan view of a double lumen pigtail catheter.

FIG. 1B is a cross-sectional view of a braided pre-bend region.

FIG. 1C is a plan view of a distal shaft segment of the double lumen pigtail catheter.

FIG. 1D is a cross-sectional view of the braided pre-bend region.

FIG. 1E is a plan view of the pigtail catheter extending into the left ventricle of a heart.

FIG. 1F is a plan view of the pigtail catheter with a coil apex extending into an aortic valve cusp nadir.

FIG. 2 is a cross-sectional view of a double lumen shaft.

FIG. 3 is a cross-sectional view of a single lumen shaft.

FIG. 4 is a plan view of a distal shaft segment showing a pigtail coil plane.

FIG. 5 is a plan view of a pigtail catheter extending into the left ventricle showing a shaft bend plane and a coil plane.

FIG. 6 is a plan view of a distal shaft segment.

FIG. 7 is a cross-sectional view through a single lumen shaft.

FIG. 8 is a plan view of an embodiment of a distal shaft segment.

FIG. 9 is a cross-sectional view of a double lumen shaft with a collapsed oval outer wall.

FIG. 10 is a cross-sectional view of a single lumen shaft formed by reflowing a collapsed oval lumen.

FIG. 11A is a plan view of a straight linear catheter used for pressure gradient measurement.

FIG. 11B is a plan view of a distal shaft segment showing a shaft curve.

FIG. 11C is a cross-sectional view of a double lumen shaft.

FIG. 12 is a plan view of a cardiac diagnostic catheter extending within an aorta.

FIG. 13 is a plan view of a double lumen pigtail catheter extending over a cardiac diagnostic catheter within the aorta.

FIG. 14 is a semi-perspective view of a cardiac diagnostic catheter and a pigtail catheter advanced across the aortic valve and into the left ventricle.

FIG. 15 is a semi-perspective view of the pigtail catheter within the left ventricle after withdrawal of the cardiac diagnostic catheter.

FIG. 16 is a semi-perspective view of a pigtail catheter extending within the left ventricle over a therapeutic guidewire.

FIG. 17A is a semi-perspective view of a pigtail catheter extending within the left ventricle to measure a pressure gradient across a stenosis due to hypertrophic cardiac muscle.

FIG. 17B is a plan view of a coil having a distal end positioned near an inner surface.

FIG. 17C is a plan view of a coil having orifices located along an inner surface of the coil.

FIG. 17D is a plan view of a coil having a distal opening in close contact with an opposing wall.

FIG. 17E is a plan view of a coil having oval orifices on an inner surface.

FIG. 18 is a semi-perspective view of the double lumen pigtail catheter positioned with a distal opening in the left ventricle and proximal orifices positioned within the aorta.

FIG. 19 is a cross-sectional view of a double lumen shaft having a wall fiber located in the outer wall.

FIG. 20 is a cross-sectional view of a double lumen shaft having an oval outer surface.

FIG. 21 is a plan view of a component model showing a pressure signal, P(t), from the heart, Inertia, Lₚ, of the fluid within the pigtail catheter system, Resistance, Rₚ, for fluid movement within the lumens of the pigtail catheter, and the total system compliance, Cₜ.

The drawing figures are not necessarily to scale. Certain features and components may be shown exaggerated in scale or in schematic form, and some details may not be shown in the interest of clarity and conciseness.

### DETAILED DESCRIPTION

While the pigtail catheters of the present invention can be used to measure a pressure gradient across a narrowing in a tubular member or chamber of the body, much of the following description will be focused on a cardiac pigtail catheter that is configured to measure a pressure gradient across a stenotic aortic valve.

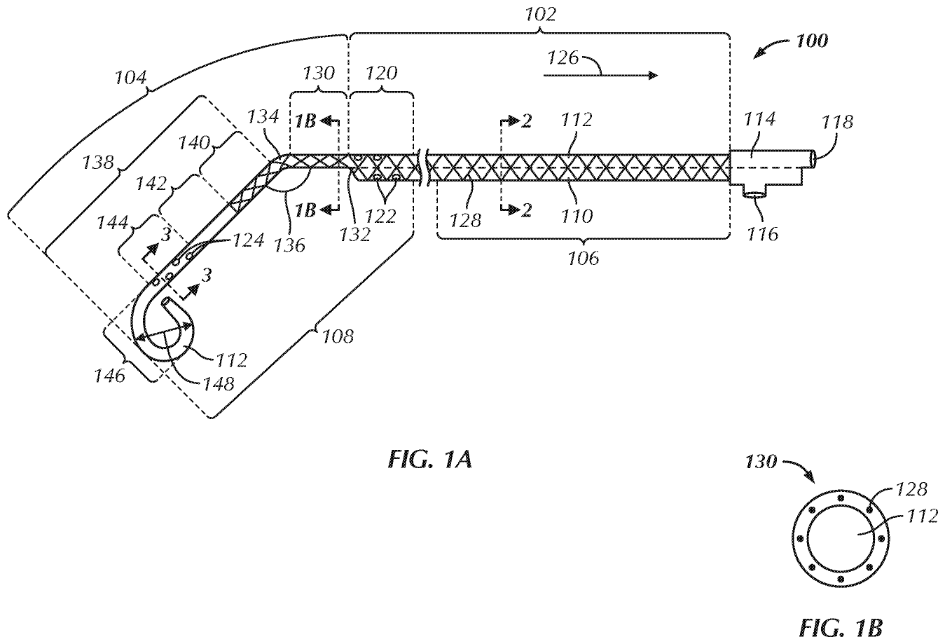

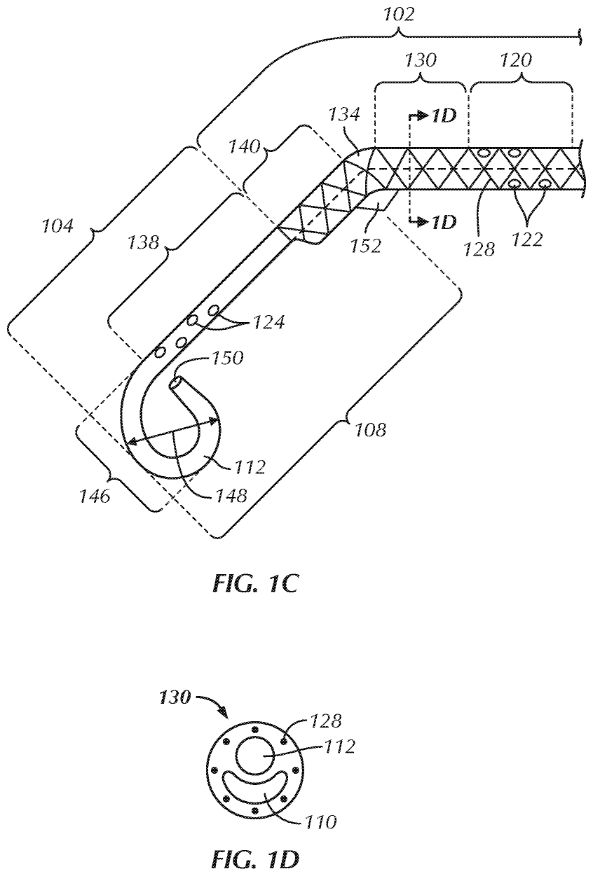

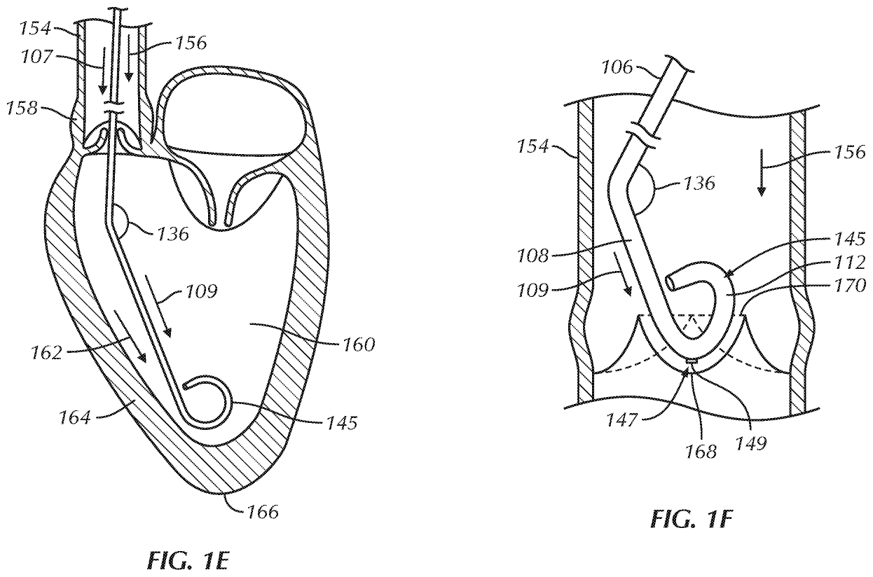

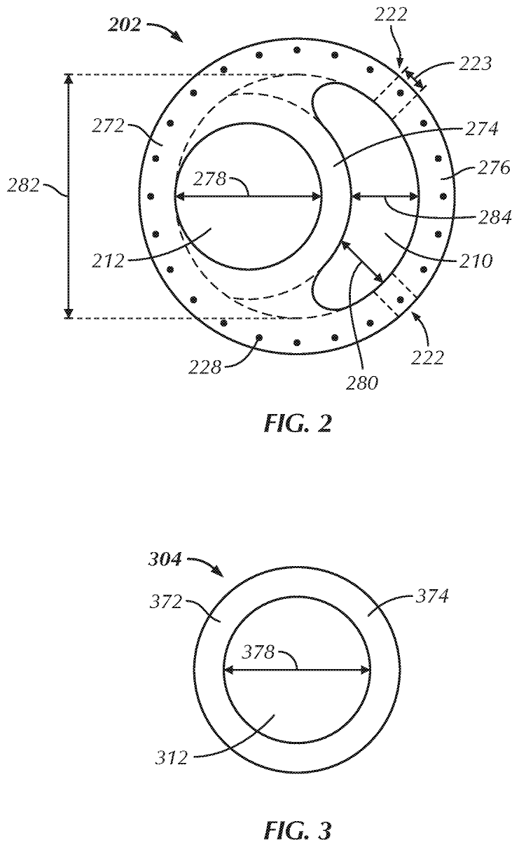

FIGS. 1A-1F, 2, 3, and 5 show an embodiment of a present pigtail catheter. The proximal shaft segment can contain two lumens that transmit pressure signals from the distal catheter segment to the proximal catheter segment via a circular distal pressure lumen that can be approximately circular in cross-section and a more oval proximal pressure lumen. The proximal shaft segment can extend from a manifold located outside the body into the vasculature of the body, in use, to reach a narrowing within a body tubular member or cardiovascular member of the body located several centimeters (range 5-140 cm, for example) from the access site of catheter entry into the body. The distal catheter segment can contain distal orifices and a distal opening that are in direct and immediate fluid communication with body fluid located distal to a narrowing. The distal catheter segment can also contain proximal orifices that are in direct and immediate fluid communication with body fluid proximal to the narrowing. The distal orifices are in direct fluid communication with the distal pressure lumen, which transmits a distal pressure (from a location distal to a narrowing (such as a stenotic aortic valve, for example, or other body member) to the distal pressure port located on a manifold at the proximal end of the catheter. The proximal orifices are in direct fluid communication with the proximal pressure lumen, which can be oval in cross-sectional shape and transmits a proximal pressure signal (from a location proximal to a narrowing in the body member) to the proximal pressure port located on the manifold at the proximal end of the catheter. The proximal pressure port and distal pressure port of the manifold can be connected to a pressure transducer to measure and record a pressure gradient across the narrowing of the tubular member of the body.

Further, the pressure ports on the catheter manifold can be used alternately for intravascular contrast injection and thus provide opacification to define structure and function of a lumen, a chamber, or a valve. Contrast can be injected into the proximal pressure port and delivered via the proximal pressure lumen to exit the proximal orifices into a vascular lumen or a chamber proximal to a narrowing. Alternately, contrast can be injected into the distal pressure port and delivered via the distal pressure lumen to exit via the distal orifices or distal opening into a vascular lumen or a chamber distal to a narrowing. It is noted that when delivering contrast via the proximal or distal pressure port, back pressure generated via a syringe or other pressure generating device (for delivering contrast under pressure) is needed to create adequate flow of contrast to opacify a specific chamber. The back pressure can cause disengagement of the pressure port from the pressure generating device if the pressure lumen diameter or orifice diameter are not of a sufficient diameter or hydraulic diameter. The hydraulic diameter of the pressure lumens, in many examples, should be at approximately 0.020 inches (range 0.018-0.038 inches, for example) to ensure that contrast delivery does not generate excessive back pressure.

For the embodiment intended for measurement of pressure gradient across a stenotic aortic valve, for example, the distal shaft segment is configured to permit entry into a multitude of arterial and venous entry sites, for example, the femoral artery access site, and reside within the aorta and the LV such that the distal shaft segment extends across the narrowing or stenosis found in the tubular or chamber-like member of the body. The distal shaft segment can be conceptually divided into several regions, the proximal orifice region, the braided pre-bend region, the shaft bend, the braided post-bend region, the linear segment, and the coil region. The coil and adjacent flexible linear region contain only a single distal pressure lumen that can transmit a distal pressure signal from a body lumen distal to the narrowing to the distal pressure port located on the catheter manifold. The post-bend region extends from the shaft bend to the distal end of the pigtail catheter. Note that the proximal pressure lumen found in the proximal shaft segment can be easily removed from or not included within the distal shaft segment; the oval lateral wall of the proximal pressure lumen can be skived away, thermally removed, or otherwise rendered incapable of transmitting an accurate proximal pressure signal from the coil or the flexible linear region of the distal shaft segment.

As shown in FIGS. 1A and 1B, the braided post-bend region and braided pre-bend region can contain a single distal pressure lumen. Alternately, as shown in FIGS. 1C and 1D, the braided post-bend region and braided pre-bend region can contain both the distal pressure lumen and the proximal pressure lumen; although the proximal pressure lumen is not necessary distal to the proximal orifice region, extending the double lumen shaft to the shaft junction located distal to the post-bend region, as shown in FIG. 1C (shaft junction defines a change from double lumen shaft to single lumen shaft) may provide benefit for ease of manufacturing. As shown in FIG. 1E, a shaft bend in the distal shaft segment of about 155 degrees included angle (range 145-160 degrees, for example) allows the distal shaft portion to extend into the generally elongated LV chamber axis of which is angulated or bent in relation to the proximal aortic central axis minimizing aggressive contact with wall segments (predominantly the inferobasal segment of the LV) with the ensuing potential for electrical arrhythmias or disruption of normal sequential contractions that are necessary for obtaining accurate pressure waveforms and optimal contrast opacification to assess LV systolic function. A shaft bend of greater than 145 degrees also allows the distal shaft segment axis to more closely align with the central aortic axis and allows a coil apex located on the distal-most curve of the coil to be placed into the noncoronary aortic valve cusp nadir to most optimally view the valve cusp nadir via fluoroscopy or ultrasound, as shown in FIG. 1F. A radiopaque marker or other marker can be placed on the coil apex for visualization purposes and provide the operator with an accurate location of the aortic valve annulus and native leaflets and potential location of where to place a TAVR device, for example. The coil can have an asymmetric shape with a coil apex having a radius of curvature of about 3 mm (range 2-4 mm, for example) that is smaller than the radius of curvature for the remainder of the coil with a radius of curvature of about 5 mm. This asymmetric coil can extend the coil apex more fully into the nadir of the aortic leaflet cusp and thereby more accurately locate the aortic annulus and more accurately properly place a TAVR device, for example.

The shaft bend angle may not be required for other embodiments of the present invention, such as for pigtail catheters used for measuring a pressure gradient across narrowings in the vasculature or in tubular members of the body other than the narrowing (e.g., between the LV and the aorta across a stenotic aortic valve). The shaft bend forms a shaft bend plane with the braided pre-bend region and the braided post-bend region on each side of the shaft bend. The braided structure of the catheter shaft can extend distal to the shaft bend for 2.0 cm (range 0.5-4 cm, for example) to reach the distal end of the braided structure to provide adequate torque transmission capabilities from the proximal shaft segment to the distal shaft segment. The length of the catheter shaft distal to the shaft bend may not extend into the apex of the LV chamber to avoid potential for pre-ventricular contractions (PVC's).

The proximal shaft segment can have a braided structure applied to the lateral walls of the double lumen tubing; the braided structure can have about a 0.004-inch diameter metallic wire or polymeric fiber, for example. The braided structure can extend into various regions of the distal shaft segment to allow the operator to apply a torque to the catheter manifold and proximal shaft segment externalized outside of the body and transmit the torque to the distal shaft segment, including regions that are located distal to the shaft bend. The regions of the distal shaft segment that can be braided include the proximal orifice region, the braided pre-bend region, the shaft bend, the braided post-bend region, and distal orifice region, as shown in FIGS. 1A and 1C; the braided structure, in some examples, is absent in the coil and immediately proximal to the coil for 4-10 mm. The braided structure can extend throughout the entire length of the distal shaft segment, but if the braided structure is present in the coil or the flexible linear region near the coil, the braided structure in that region should be formed from very thin and easily bent fibers such that disruption of the cordae tendineae, for example, is not caused by entanglement with the coil during removal or repositioning of the pigtail catheter within the heart, vascular, or non-vascular structures. In many examples, the braided structure should not exceed a fiber diameter of approximately 0.010 inch diameter due to the potential for creating excessive profile for the catheter shaft due to crossover of the braided fibers; the braided fiber or wires should preferably be less than about 0.005 inches in diameter; the proximal catheter shaft profile that is consistent with the dimensions and pressure transmitting capability may have an external profile as low as 6 French (F) or less, although a larger profile double lumen pigtail catheter ranging from 7 F-10 F can be acceptable for specific therapeutic procedures including some TAVR procedures, for example, where a large profile introducer sheath is necessary to deliver the therapeutic catheter.

One or more proximal orifices located in the pre-bend region about 3 cm (range 2-8 cm, for example) proximal to the shaft bend and above the sinotubular ridge of the aorta provide fluid communication and aortic pressure transmission from the aorta to the oval proximal pressure lumen and further signal transmission to the proximal pressure port, for example, located on the manifold. Positioning the proximal orifices above the sinotubular ridge and proximal to the shaft bend can avoid inaccurate measurement of the pressure gradient across a stenosis; such inaccurate measurement can result from lack of pressure recovery downstream of the stenosis due to placement of the proximal orifices too close to a vena-contracta jet associated with blood flow through stenotic valve leaflets. A pressure transducer connected to the proximal pressure port and distal pressure port of the manifold can simultaneously measure a pressure difference between the proximal pressure port and the distal pressure port thereby measuring a pressure gradient across the narrowing, for example, of an aortic valve or other stenosis found in the chambers of the heart or other vascular lumen, non-vascular lumen, or chambers of the body.

The proximal orifice holes can be located between openings of the braided structure, which provide individual braided fibers that are spaced apart (between fibers of the braided structure) to allow about four (range approximately 1-8, for example) 0.020-inch diameter (range 0.018-0.028 inches, for example) proximal orifices to be placed into the proximal shaft portion. The proximal orifice holes can be in fluid communication with an oval proximal pressure lumen, which can have a major diameter of about 0.035 inches and a minor diameter of about 0.016 inches; the hydraulic diameter of this oval or elliptical proximal pressure lumen can be about 0.020 inches (range 0.018-0.025 inches, for example) to ensure fully accurate transmission of the pressure signal from the aorta and accurate determination of the pressure gradient across the aortic valve. The hydraulic diameter for the oval proximal pressure lumen is determined from the equation: DH=(4BC(64−16E²))/((B−C)(64−3E⁴)), where 2B is the major diameter, 2C is the minor diameter, DH is the hydraulic diameter, and E=(B−C)/(B+C).

The double lumen shaft can be formed, for example, from polyurethane, Pebax (block copolymers composed of rigid polyamide blocks and soft polyether block and sold by Arkema), polyethylene, or other polymer that is extrudable, thermally reformable, and found in medical catheter devices; the polymer should be resilient and preferably soft enough such that properties of the double lumen shaft and single lumen shaft can be obtained (but not necessarily obtained) from a single shaft extrusion for both the double and single lumen shafts, if possible, with a thermal post-extrusion step to form the single lumen shaft. The double lumen shaft should at least allow thermal or other joining process to be performed, if necessary, to join the double lumen shaft to the single lumen shaft.

The double lumen shaft extends to a shaft junction distal to which the single lumen shaft extends distally containing a single (only one lumen that is able to provide adequate pressure transmission capabilities) distal pressure lumen that is able to provide adequate pressure transmission of a distal luminal pressure to a distal port located on the catheter manifold, as shown in FIGS. 1A, 1C, and 3. The distal shaft segment can contain a flexible linear region that extends for about 4 cm (range 2-6 cm, for example) and may not contain a braided structure to a pigtail coil of the distal shaft portion. The flexible linear region can provide a shaft region with intermediate torque transmitting characteristics and intermediate bending stiffness between the braided post-bend region and the coil region that can extend into the LV, for example, without causing electrical disturbance due to interaction with certain LV wall segments. The distal orifices located proximally and adjacent to the coil allow the simultaneous measurement of pressure distal to the narrowing with the measurement of pressure proximal to the narrowing via the proximal orifices; the measurement of such simultaneous proximal and distal pressures is indicative of the absolute pressure gradient across the narrowing without potential for error that can be caused by the presence of a high velocity jet across the narrowing and the resulting lowering of a localized pressure reading near the jet. The coil can have a coil diameter of about 1 cm (range 7 mm-15 mm, for example) and have a distal opening that provides both a passage for a guidewire such as a 0.035-inch guidewire, for example, and provides fluid communication with the distal pressure lumen for transmission of a distal pressure signal from the LV to distal pressure port located at the catheter manifold. The distal opening diameter along with the distal pressure lumen diameter can be formed with a smaller diameter of about 0.025-0.032 inches, for example, to accommodate a smaller diameter guidewire and thereby allow the present invention to have a profile as low as 5 F-6 F for applications that can accommodate a less supportive guidewire. The guidewire can extend through and proximally from the distal pressure port (or guidewire port), throughout the distal pressure lumen, and through and distal from the distal opening of the pigtail catheter.

The flexible linear region can also have distal shaft orifices located just proximal to the floppy and more flexible pigtail coil which can be located on the single lumen shaft, and the distal orifices extend over an axial length of the single lumen shaft of about 1 cm (range 5-20 mm, for example). One or more distal orifices (about 4 orifices; range of approximately 1-6, for example) can extend within the coil and up to about 2 cm (range 1-4 cm, for example) adjacent and proximal to the coil and can be located within the coil; the orientation of the distal orifices should be circumferential around the catheter shaft both proximal to the coil and within the coil. The distal orifices have a diameter of about 0.020 inches (range 0.018-0.028 inches, for example) to provide adequate transmission of a pressure signal from the LV to the distal pressure port located on the manifold. The single lumen shaft can be formed from polyurethane, Pebax, polyethylene or other polymer that can be extruded and formed into a coil shape and retain its shape resiliently.

The distal coil should be soft enough to allow straightening of the pigtail coil and the distal shaft segment when passing over a guidewire and should be able to return to the coiled shape once the guidewire has been removed. The coil can be formed from a soft polymeric material that will unfold with a force of about 25 grams or less to ensure that cordae tendineae, for example, when entrapped by the coil are not stretched or torn. The coil can have a round shape, as shown in FIGS. 1A and 1C, or the coil can have an oval shape, oblong shape, other geometrical shape, or in some instances the coil can be omitted from the catheter configuration and a simple straight distal shaft segment can be employed rather than having a coil present. The oval shaped coil shown in FIG. 1E has a smaller radius of curvature (i.e., smaller than the remaining portions of the coil) in the coil apex found in the distal-most portion of the oval-shaped curved coil to most optimally allow the coil apex to locate in the nadir of the native leaflet cusp. Once the pigtail catheter has been delivered into the LV, for example, over a guidewire that is contained within the distal pressure lumen, the guidewire is intended to be removed prior to measuring LV pressure via the distal pressure lumen. The distal pressure lumen has a diameter able to deliver a 0.035-inch guidewire, for example, and hence has a hydraulic diameter (the hydraulic diameter is equal to the diameter of a circular lumen) that is at least 0.035 inches and preferably 0.002-0.004 inches larger in diameter than the guidewire diameter to provide ease of guidewire movement.

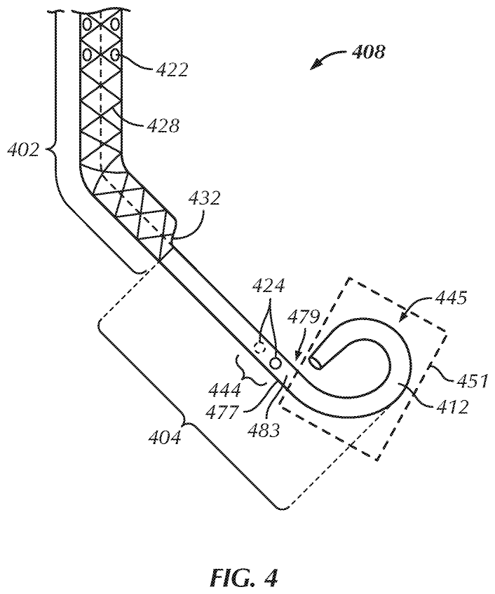

As shown in FIGS. 1C, 2, 3, and 4, the braided structure of the double lumen shaft can end at or near the junction with the single lumen shaft. The single lumen shaft can be formed by skiving away, cutting away, or thermal removal of the oval lateral wall of the proximal pressure lumen thereby leaving the distal pressure lumen contained by the common wall and lateral circular wall to formulate the distal lumen of the distal shaft portion. The oval lateral wall can also be thermally melted or otherwise attached to the common wall to form a single lumen shaft; a mandrel can be placed within the distal pressure lumen to preserve the shape and dimension of the distal pressure lumen during such thermal reforming process. Distal orifices can be formed through the wall of the single lumen shaft with openings located in the distal orifice region on the inner curve surface, outer curve surface, and on the in-plane surfaces (see FIG. 4) located in the pigtail coil plane distributed circumferentially along the coil plane to minimize the potential for kinking in the distal orifice region and to provide for unimpeded access of the body fluid into direct contact with an open distal orifice, for example, to accurately represent the pressure within the body fluid.

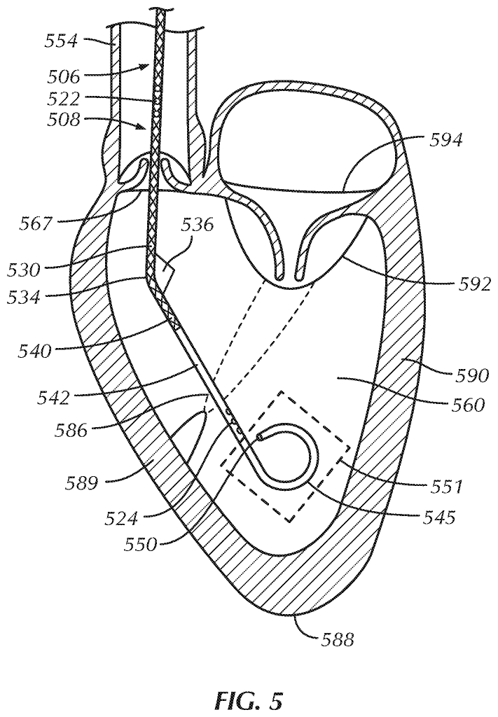

When the pigtail catheter of the present invention is used to measure a pressure gradient across the aortic valve, for example, as shown in FIG. 5, the coil along with the distal pressure orifices can be positioned in the LV and the proximal pressure orifices can be positioned in the ascending aorta. The coil plane can be further angulated in a second plane relative to the shaft bend plane to form a coil plane angulation that is leftward of the intraventricular septum when viewed anteriorly from the frontal plane; the angulation is directed toward the anterolateral free wall of the LV. This coil plane angulation allows the coil to reside within the LV without impinging upon the inferior or septal wall segments of the LV and minimizing ventricular ectopy and optimizing LV opacification during left ventriculography by injecting contrast at the mitral inflow as opposed to the left ventricular apex; this mitral inflow position opacifies the LV more uniformly with less contrast and this enhances imaging yields a more optimal assessment of LV systolic function. The coil plane angulation describes the angular separation between the coil plane angle and the shaft bend plane; the coil plane angulation may be about 30 degrees (range 5-45 degrees, for example).

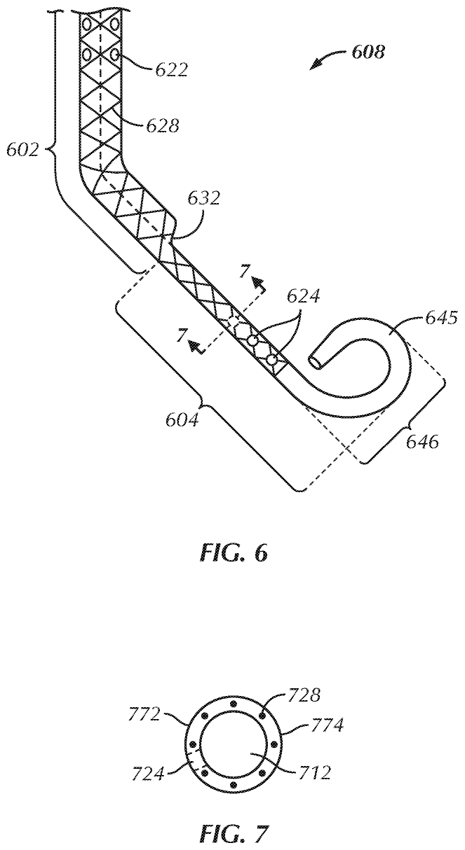

Alternately, as shown in FIGS. 6 and 7, the braided structure found in the double lumen shaft can be extended into a portion of the single lumen shaft, up to the location of the pigtail coil, for example. The presence of such a braid in the single lumen shaft can further ensure that the single lumen shaft is able to resist kinking and to transmit a torque to the coil via an application of torque to the catheter shaft by the operator at an exteriorized proximal shaft segment and manifold. The coil should be able to uncoil with a force of less than about 25 grams to ensure that cordae tendineae, for example, are not torn or ruptured.

To form such a catheter shaft, the skiving of the oval lateral wall of the oval proximal pressure lumen can occur prior to placement of a braid over both the double lumen shaft and single lumen shaft. Axial extension of the braided structure in the single lumen shaft would place the braided structure into intimate contact with the wall of the single lumen shaft. Subsequent thermal reflow of the braided material into the outer wall of the catheters shaft by application of heat and an outer shrink wrap that applies an inward force onto the braided structure, and further by protecting the proximal pressure lumen and distal pressure lumen with shaped mandrels (that match the lumen shapes), such as Teflon (a synthetic fluoropolymer of tetrafluoroethylene made by Chemours) mandrels, for example, can form the braided catheter shaft. The reflow of polymer shaft material can allow the braid to penetrate the circular lateral wall and oval lateral wall of the double lumen shaft and allow penetration into the circular lateral wall and common wall of the double lumen shaft, as shown in FIG. 7. Alternate methods of forming the braided proximal and distal shaft portions are contemplated using reflow techniques known in the catheter manufacturing industry.

Even further alternately, as shown in FIGS. 8-10, an elastic member can be inserted within a collapsed proximal pressure lumen at a location just proximal to the shaft junction and extending within a thermally reflowable collapsed oval lumen distal to the shaft junction and potentially extending within the flexible linear region up to the coil. The elastic member can be a flat ribbon of Nitinol, for example, that is formed into the shape of the shaft into which it is to be placed. The elastic member can be a flat ribbon of approximately 0.003-inch thickness (range 0.002-0.007 inches, for example) and having a width of approximately 0.5 mm (range 0.1-2 mm, for example). The elastic member can help to prevent kinking at the junction. The coil can be preferably formed with a soft polymeric plastic without the presence of the elastic member to ensure that the coil is able to uncoil with a force of less than 25 grams to protect cordae tendineae from disruption.

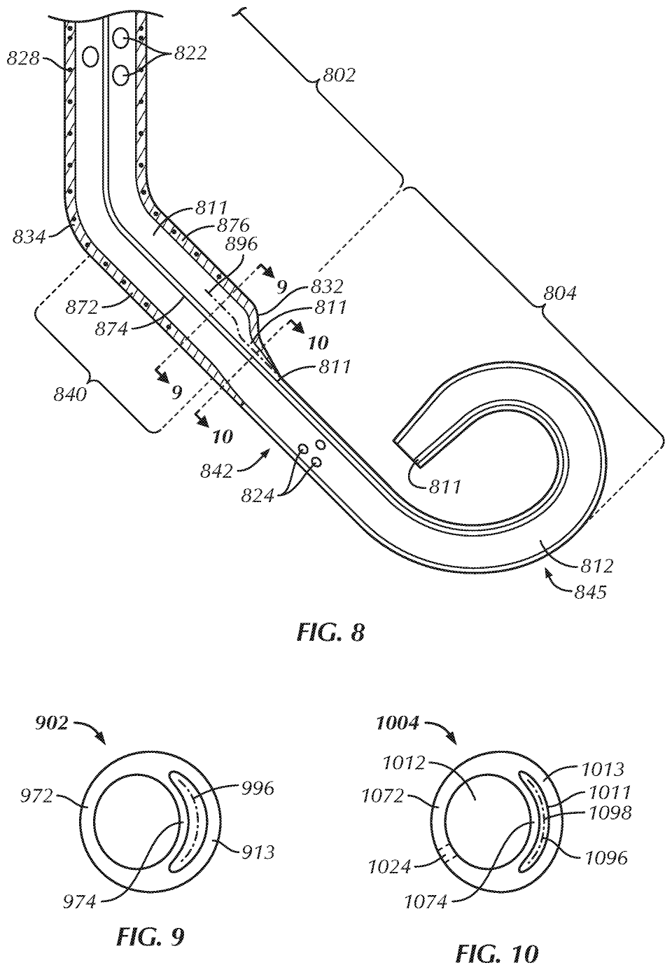

To form this distal shaft portion, a braid can be located only within the double lumen shaft, as shown in FIGS. 1C and 9. The elastic member can be slid into the distal oval opening and placed such that it overlaps the shaft junction by about 10 mm (range is approximately 5-25 mm, for example) into the braided region of the double lumen shaft and into the reflowable oval lumen of the single lumen shaft. A portion of the double lumen shaft distal to the shaft junction (or alternately, also proximal to the shaft junction in the braided post-bend region) can then be thermally reflowed to cause the oval proximal pressure lumen to collapse and entrap the elastic member and form an attachment between the elastic member, the common wall and the collapsed oval lateral wall, as shown in FIG. 10; the double lumen shaft has therein been converted to a single lumen shaft distal to the shaft junction. The oval lateral wall can be thermally melted into contact with the common wall to form a single lumen shaft distal to the elastic member; alternately the oval lateral wall can be skived away to form a single lumen shaft distal to the elastic member. Distal openings can be formed into the central lumen at locations that do not interfere with or are blocked by the presence of the elastic member.

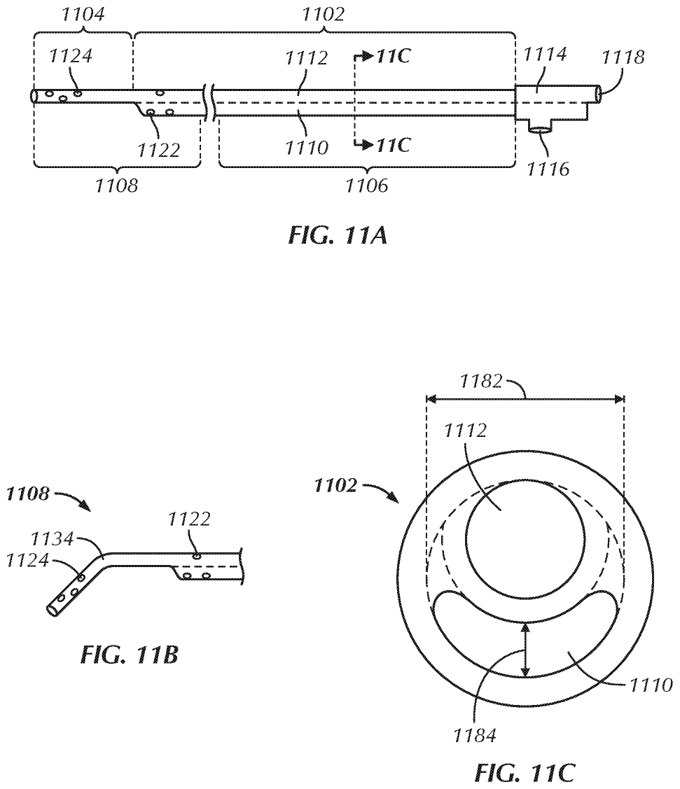

FIGS. 11A and 11B show another embodiment for the present invention that is directed to measuring a pressure gradient across a narrowing within a linear tubular member, chambers, or cavities of the body and across a narrowing within the cardiovascular or non-cardiovascular anatomy of the body. In this embodiment, the distal shaft segment that is placed across the narrowing can have a straight configuration, as shown in FIG. 11A; alternately, as shown in FIG. 11B, the distal shaft segment can have a shaft curve located near the distal end. The shaft curve can assist in traversing across a tortuous or curved path or for entering a side branch of a tubular member. The proximal catheter shaft can be braided to assist with torque transmission from the manifold to the distal shaft segment to aid in traversing the vasculature, for example, and providing push characteristics to the shaft without affecting flexibility significantly. The catheter can have a distal pressure lumen of 0.025 inches (range 0.018-0.038 inches, for example) that can provide passage for a guidewire and providing for transmission of a non-attenuated pressure signal; the smaller 0.018-inch distal pressure lumen can be capable of transmitting an accurate and non-attenuated pressure signal back to the distal pressure port located on the manifold. The distal orifices can have a diameter of approximately 0.020 inches (range 0.018-0.028 inches, for example). As shown in FIG. 11C, the proximal pressure lumen can have a major diameter of approximately 0.028 inches and a minor diameter of 0.016 inches to provide a hydraulic diameter of at least 0.018 inches and can transmit with adequate accuracy a pressure signal back to the proximal pressure port on the manifold. The proximal pressure orifices can have a diameter of approximately 0.020 inches (range 0.018-0.028 inches, for example). This catheter formed from a double lumen construction as described in earlier embodiments allows improved pressure transmission signals along a catheter having a length of up to 140 cm from the distal shaft segment back to the pressure ports located on the manifold than currently used dual lumen catheters formed from concentric tubes. The profile of the catheter of this embodiment can be as small as 4.5 F-6 F.

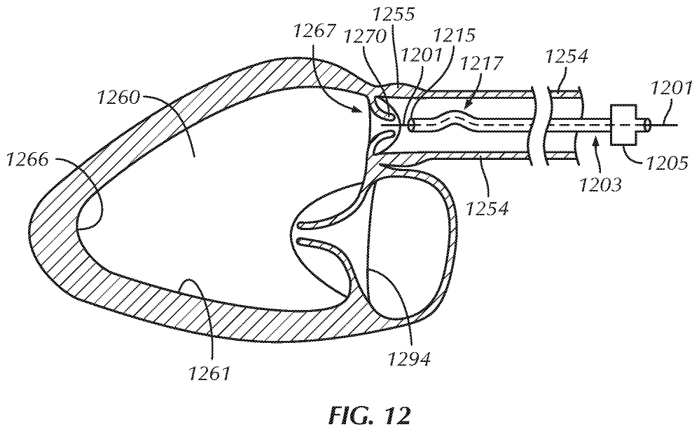

A standard procedure for advancing a straight-tip guidewire and diagnostic cardiology catheter across a stenotic aortic valve is described along with its limitations. One diagnostic cardiology catheter, for example, that is currently being used to deliver a straight-tip guidewire, for example, from the aorta to the LV across a stenotic aortic valve is a single lumen Amplatz Left (AL) catheter such as that shown in FIG. 12; the profile of such catheters can range from 4 F-8 F, for example, and can be delivered over a guidewire with diameters ranging from 0.025 inches to 0.038 inches. The distal portion of the cardiac diagnostic catheter can be positioned within the aortic root while the straight-tip guidewire is advanced within a thru-lumen across the stenotic aortic annulus and into the LV under fluoroscopic guidance. The cardiac diagnostic catheter can then be advanced over the straight-tip guidewire and positioned in the LV. Upon removal of the straight-tip guidewire, the cardiac diagnostic catheter can be used to provide a passage for delivery of a specialized guidewire which is supportive and has a coiled shape positioned in the distal LV to prevent LV perforation.

It should be noted that the cardiac diagnostic catheter can have a distal end configuration which has a tip generally pointed toward the LV apex. This configuration may result in a straight-tip wire being inadvertently forcefully advanced into the LV apex when advancing the cardiac diagnostic catheter into the LV resulting in perforation. The circular lumen of a pigtail embodiment of the present invention can accommodate a low profile cardiac diagnostic catheter, which can be used to exchange the pigtail catheter mitigating the risk of exchange of catheters over the straight tip guidewire only.

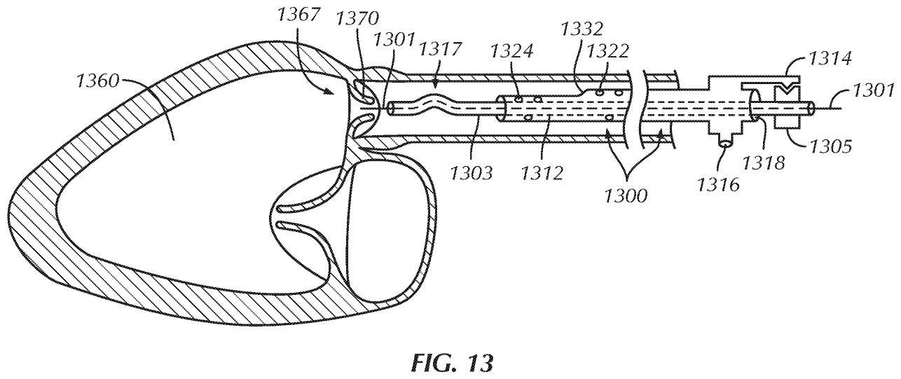

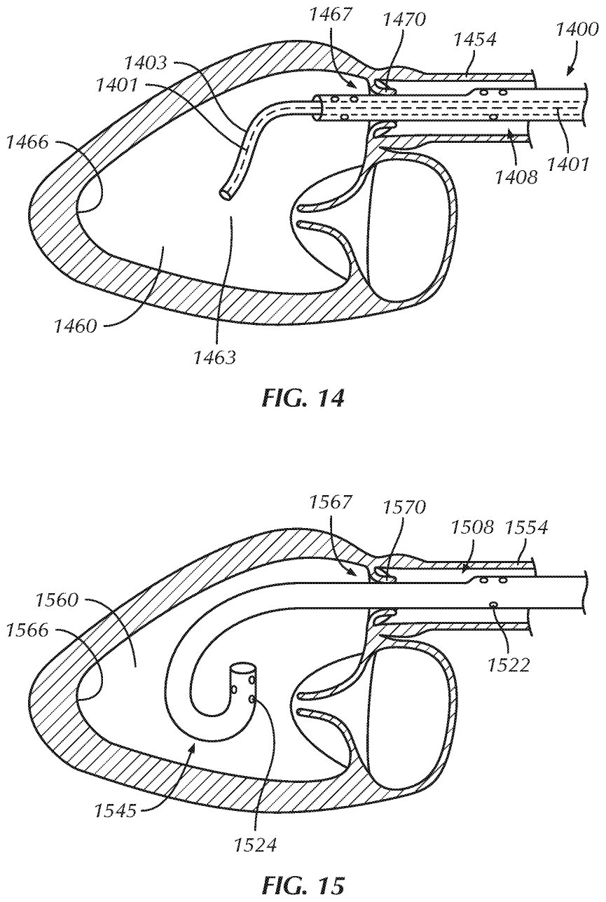

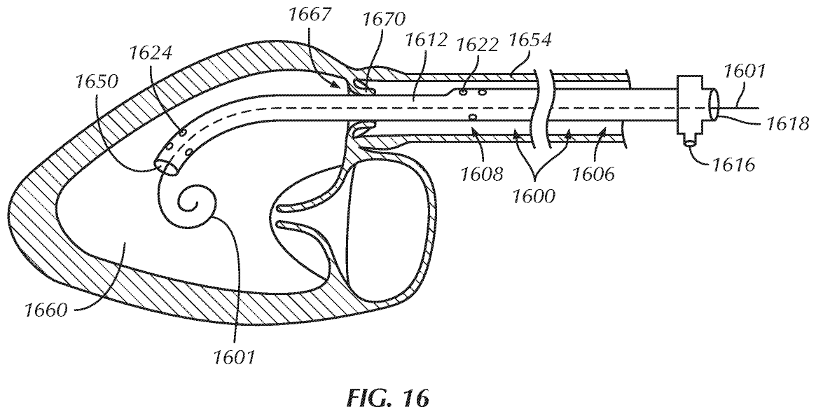

As shown in FIGS. 13-15, the double lumen pigtail catheter of the present invention can be front-loaded over the outside surface of a cardiac diagnostic catheter with the cardiac diagnostic catheter shaft residing within the distal lumen of the pigtail catheter. The distal portion of the cardiac diagnostic catheter can extend distally beyond the distal opening of the pigtail catheter by 10 cm (range 8-15 cm, for example) such that shape of the cardiac diagnostic distal portion is not significantly affected by the shape of the pigtail coil and can effectively direct the straight-tip guidewire through the stenotic aortic leaflets, as shown in FIG. 13. The cardiac diagnostic catheter can be advanced over a fixed guidewire which is then partially retracted into the cardiac diagnostic catheter, as shown in FIG. 14. With the guidewire fully withdrawn, the cardiac diagnostic catheter can provide a less traumatic rail to pass the pigtail catheter over an into the LV. Upon removal of the cardiac diagnostic catheter, the pigtail catheter can be located within the LV chamber, as shown in FIG. 15.

The pigtail catheter can have a distal lumen diameter of 0.052 inches (range 0.045-0.060 inches), for example, to accommodate passage of a 4 F cardiac diagnostic catheter (range 3.5 F-4.5 F, for example); the pigtail catheter can be formed with an overall profile of 8F while maintaining hydraulic diameters for the proximal and distal lumens greater than or equal to 0.018 inches and providing highly accurate pressure signal transmission. Alternately, the pigtail catheter can have a distal lumen diameter of 0.069 inches (range 0.065-0.075 inches), for example, to accommodate passage of a 5 F cardiac diagnostic catheter (range 4.5 F-5.5 F, for example) that is able to follow over a 0.032-inch guidewire, for example; the pigtail catheter can be formed with an overall profile of 9-10 F while maintaining hydraulic diameters for the proximal and distal lumens greater than 0.018 inches and providing highly accurate pressure signal transmission. Further alternately, the pigtail catheter of the present invention can track directly over a 0.035-inch guidewire with a profile for the pigtail catheter of 7 F-8 F and with highly accurate pressure signal transmission.

A J-tipped guidewire can be advanced leading a cardiac diagnostic catheter via a vascular access sheath and advanced into the aortic root. A straight-tip guidewire can then be exchanged for the J-tipped guidewire to be advanced across the stenotic aortic valve and advanced into the LV.

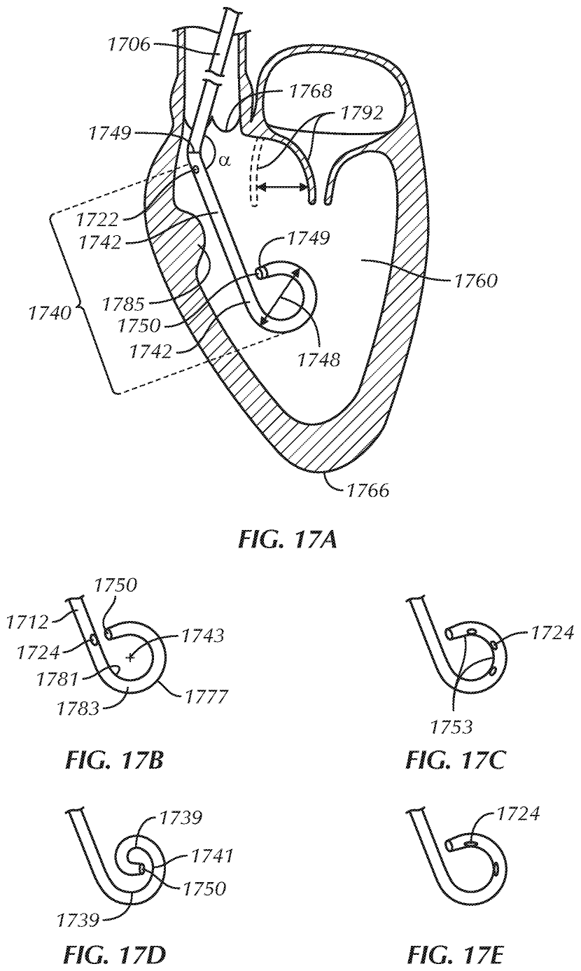

FIG. 17A shows the distal shaft segment of the double lumen pigtail catheter of the present invention placed within the LV of a patient with hypertrophic obstructive cardiomyopathy (HOCM). The HOCM pigtail catheter can have a shaft that is braided near the bend angle and in other portions of the proximal shaft segment to provide for torque control of the distal shaft segment and to prevent kinking of the catheter shaft. The coil and flexible linear region of the post-bend region generally may not be braided to provide a more flexible catheter shaft that does not cause potential harm to the cordae tendineae within the LV. The bend angle of 155 degrees (range 145-165 degrees, for example) can allow the catheter to be delivered into the LV without making forceful contact with the inferobasilar wall of the LV, which can lead to ectopy. The 155-degree bend angle can also allow a straighter alignment of the of the distal catheter shaft with the ascending aorta to allow the coil apex and a radiopaque marker located on the coil apex to seat more completely into the nadir of the aortic cusp nadir and allow more accurate visualization of the aortic valve cusp and location of the aortic annulus for accurate placement of a TAVR device, for example. The coil can have a radiopaque marker located at its distal opening to accurately identify the location of the distal pressure measurement.

The coil can have a coil diameter of about 5 cm (range 3-8 cm, for example) which is smaller than the coil diameter for other cardiac pigtail catheter coils of the present invention. The smaller 5 mm coil diameter is better suited to not interfere with the systolic contraction of the LV in a HOCM patient that often has direct contact of the LV opposing walls near the LV apex during systolic contraction. During systolic LV contraction, the orifices located along the coil, or the distal opening found at the end of the coil can be blocked by the myocardial tissue. Normally such a distal opening can have a diameter of 0.038 inches (range 0.025-0.040 inches, for example) to allow passage of a guide wire and to allow transmission of pressure signal to the distal lumen of the pigtail catheter. Such orifice or distal opening blockage can result in diminution of the pressure signal that is normally transmitted through the orifices or distal opening to the distal lumen of the pigtail catheter shaft. This reduced pressure signal can result in an inaccurate measurement of the pressure within the LV. As shown in FIG. 17A, the distal opening can be positioned near (e.g., within 1 mm) or in direct contact with the catheter shaft of the flexible linear region of the post-bend shaft region. The proximity of the distal opening near the catheter shaft can prevent myocardial tissue from entering the distal opening during systolic contraction of the heart. A radiopaque marker can be positioned at the distal-most aspect of the coil to provide visualization of the coil location within the LV; the coil can be maintained at about 2-3 cm from the LV apex; the LV apex typically has more myocardial compression via opposing wall; maintaining the coil at a distance from the LV apex can be a preferred position for the coil of the HOCM pigtail catheter.

Orifices can also be placed at other locations along the coil of the HOCM pigtail catheter, as shown in FIGS. 17B-17E. As shown in FIG. 17B, an orifice can be located along the inner surface of the coil at a location at or near (within 1 mm) the distal opening. An orifice located along the inner surface of the coil can have an orifice diameter of 0.020 inches (range 0.018-0.028 inches, for example) to ensure that the pressure signal from the pressurized blood in the LV is not attenuated as it is transmitted to the distal lumen of the HOCM pigtail catheter. Alternately, a series of orifices (range 2-5 orifices, for example) can be located along the inner surface of the coil closest to the coil center without have any orifices located on the planar surface (i.e., the surface of the coil that is formed by a plane that touches along the entire coiled length of the coil) or on the outer surface of the coil, as shown in FIG. 17C. The presence of an orifice on the planar surface or the outer surface can allow myocardial tissue to penetrate such orifice and block the distal lumen and result in an inaccurate pressure signal being transmitted to the distal lumen. As shown in FIG. 17D, the coil can fold inward to form a coil of greater than 180 degrees of curvature. The distal opening can thereby be protected by coming into direct contact or near contact with an opposing wall of the coil plus be protected by the inner surfaces of neighboring regions of the coil that can assist in preventing myocardial tissue from impinging upon the distal opening. Further, the cross-sectional shape of the orifices can be oval opening in the wall of the coil, as shown in FIG. 17D; the oval orifices can have a major axis along the inner surface perimeter of the coil of about 0.030 inches (range 0.020-0.035 inches, for example) and a minor axis perpendicular to the major axis of about 0.020 inches (range 0.018-0.025 inches, for example) to provide accurate pressure signal transmission across the coil wall.

The profile of such a HOCM pigtail catheter that has a distal lumen able to follow a 0.035 guidewire and provide a proximal and distal pressure lumen with a hydraulic diameter of at least 0.018 inches (to provide accurate pressure signal transmission through the proximal and distal pressure lumens) is about 6 F-7 F.

About one or two proximal orifices are placed along the distal shaft segment at a location at or about 1 mm below a radiopaque marker that is placed at the shaft bend. The proximal orifice is intended to measure the pressure at a location between the aortic annulus (or stenotic aortic leaflets) and the hypertrophic cardiac muscle which can be separated by only 5 mm (range 3-8 mm, for example). A single orifice or two orifices positioned at this location cannot extend either downstream past the stenotic native leaflets or upstream from the hypertrophic proximal muscle and therefor this proximal orifice region cannot extend over more than 2 mm.

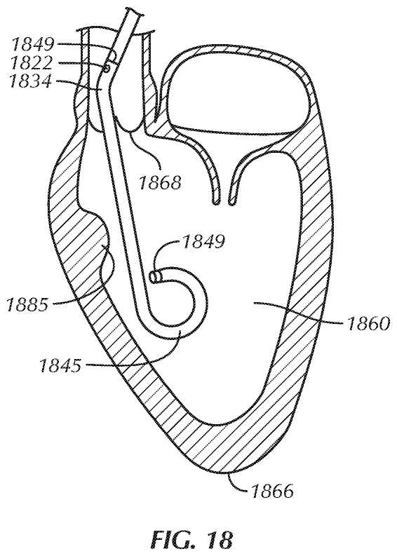

During measurement of the pressure gradient from the LV across the hypertrophic proximal muscle, the proximal orifice region can be positioned between the aortic valve and the hypertrophic cardiac muscle, and the coil can be positioned about 2-3 cm into the LV chamber from the LV apex; this positioning avoids excessive compression of the HOCM pigtail coil by systolic compression near the LV apex. The HOCM pigtail catheter can also be repositioned such that the proximal orifice region is located downstream of potentially stenotic aortic valve leaflets in the ascending aorta, as shown in FIG. 18. The coil can be located distal to the hypertrophic septal muscle such that the pressure gradient is reflective of the overall pressure gradient from the LV chamber to the aorta. The difference between the overall pressure gradient and the gradient across the hypertrophic cardiac muscle can provide a determination of the pressure gradient across the stenotic aortic valve. Based upon knowledge of the major flow resistance, treatment of stenotic aortic valve via valve replacement, for example, or the hypertrophic septal muscle treatment can occur via alcohol ablation, for example.

A second shaft bend can be placed into the pigtail catheter shaft thereby providing a proximal shaft bend with a proximal bend angle and a distal shaft bend with a distal bend angle, as shown in FIG. 18. Both the proximal and distal bend angles can be about 165 degrees and the proximal orifice region can be located just distal to the proximal orifice bend where a radiopaque marker is located. During measurement of the overall pressure gradient, the proximal orifice region can be retracted into the ascending aorta and the distal shaft bend places the coil away from the inferobasilar wall of the LV, as shown in FIG. 18. During measurement of the pressure gradient across the hypertrophic cardiac muscle, the proximal orifice region can be located below the aortic annulus and the combination of both proximal and distal shaft bends assist to place the coil away from the inferobasilar wall of the LV.

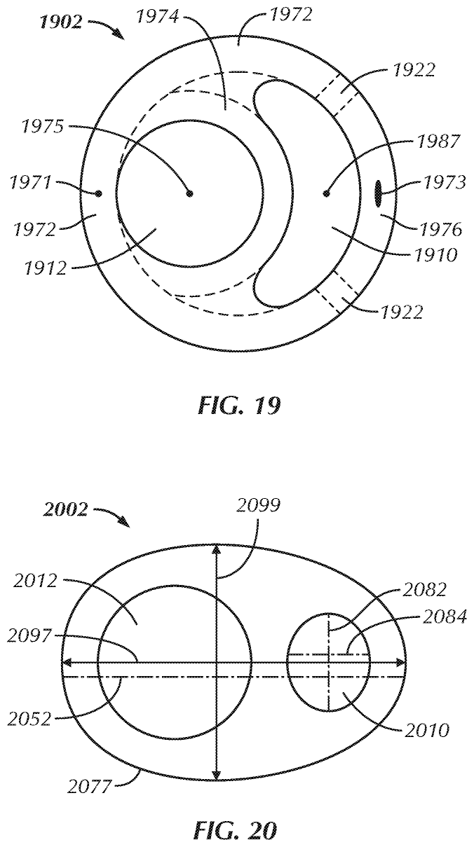

FIG. 19 shows the double lumen shaft of the present invention without a braided structure contained within the outer wall. The outer wall of this embodiment contains an oval lateral wall fiber located in the oval lateral wall in line with the center of the distal pressure lumen center and the proximal pressure lumen center. The oval lateral wall fiber can extend along the axial direction of the outer wall of the double lumen shaft. The oval lateral wall fiber can be a ribbon, a bar, or a fiber formed of a material that can easily bend but does not stretch or extend due to a relatively high tensile strength; such materials include polymeric fibers such as polyethylene terephthalate, Dacron, Kevlar, and other high tensile strength polymers; oval lateral wall fiber materials also include metal fibers such as stainless steel, Nitinol and other metals that can be formed into high tensile strength fibers that are used in medical devices. The oval lateral wall fiber should have a small thickness or radial dimension in the radial direction within the outer shaft wall of about 0.002 inches (range of 0.0005-0.005 inches, for example) such that the oval lateral wall fiber can be formed into the polymeric lateral wall of the double lumen shaft during extrusion or during a thermally based or adhesive based post-processing step. The oval lateral wall fiber can be round in cross-section or can have a rectangular cross-sectional shape of a ribbon with a width in the direction of the circumference of the outer wall of about 0.003-0.010 inches (range 0.002-0.030 inches, for example) to provide strength to prevent axial stretching yet still allow bending of the double lumen shaft to occur in a plane that is perpendicular to a line extending from the distal pressure lumen center to the proximal pressure lumen center. With a guidewire positioned within the distal pressure lumen, the double lumen shaft can bend along a plane that is perpendicular to a line extending from the distal lumen center to the proximal lumen center. This bending direction will not cause the minor diameter of the proximal pressure lumen to reduce in dimension during bending and will tend to enhance or enlarge the proximal lumen minor diameter during bending. The hydraulic radius of the proximal pressure lumen will not be reduced due to shaft bending and will be maintained at a hydraulic diameter of at least 0.018 inches. Bending of the double lumen shaft will thereby not hurt the fidelity of pressure signal transmission from the proximal orifices and through the proximal pressure lumen to the pressure transducer located at or near the manifold.

In an alternate embodiment, a circular lateral wall fiber can be place into the circular lateral wall in addition to the oval lateral wall fiber. The circular lateral wall fiber can be placed into the circular lateral wall at a location in line with the distal pressure lumen center and proximal pressure lumen center. The circular lateral wall fiber can be formed into the circular lateral wall during the extrusion process or can be formed via an alternate post processing method. The circular lateral wall fiber can have the same material and dimensional characteristics as the oval lateral wall fiber. The presence of both the circular lateral wall fiber and the oval lateral wall fiber ensures that the double lumen shaft will bend along a plane that is perpendicular to a line that joins the circular lateral wall fiber with the oval lateral wall fiber.

Other methods of placing either the oval lateral wall fiber or the circular lateral wall fiber have been contemplated; an oval lateral wall fiber can be inserted, for example, within a proximal pressure lumen and attached at a desired location along the luminal surface of the proximal pressure lumen via an adhesive, thermal bonding method, or other attachment methods. The oval lateral wall fiber can alternately be attached along the outside of the outer wall to provide similar bending characteristics to the double lumen shaft.

FIG. 20 shows another embodiment for the double lumen shaft construction with an oval double lumen shaft. In this embodiment, the oval double lumen shaft can be formed with an oval outer surface such that the oval double lumen shaft has a shaft major diameter that is 20% (range 10-30%, for example) greater than the shaft minor diameter. The shaft minor diameter can be at least 10% smaller than the shaft major diameter. A 20-25% reduction in the shaft minor diameter can provide the oval double lumen shaft with a perimeter that is 10-15% less than a round double lumen shaft and hence can fit within an introducer catheter that is about 10-15% smaller in profile. Alternately, the oval double lumen shaft can enable a larger diameter proximal pressure lumen with a better fidelity signal transmission and fit within a smaller introducer catheter profile than a round double lumen shaft will allow.

The oval double lumen shaft tends to bend in a plane that is perpendicular to the major axis. The double lumen shaft is often subjected to bending as it extends along its pathway within the body; bending of the double lumen shaft will cause the proximal pressure lumen to enlarge in a direction along the proximal lumen minor axis. Hence the proximal pressure lumen can be formed with an oval shape with the proximal lumen major axis being larger than the proximal lumen minor axis that extends in-line with the catheter shaft major axis. Bending of the double lumen shaft will then result in enlarging the proximal lumen minor axis and will result in enhanced fidelity of transmitted signal as the proximal pressure lumen becomes more rounded and results in a larger hydraulic diameter. The presence of an oval lateral wall fiber or circular lateral wall fiber can be applied to the oval double lumen shaft at locations like that described for the round double lumen shaft to enhance the tendency for the double lumen shaft to bend in a plane that is perpendicular to the shaft major axis.

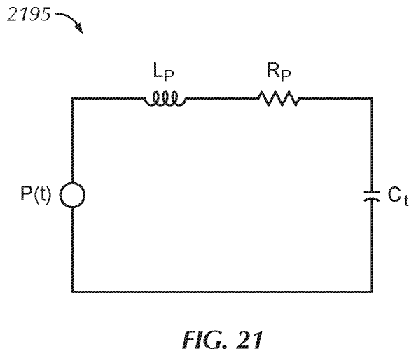

FIG. 21 shows a simplified component model of a system having a pressure signal that is transmitted through the proximal pressure lumen, for example, the pressure signal transmission being affected by multiple system characteristics. The model includes a pressure signal that represents the pressure generated by the heart pressure pulse signals. The inertia of the fluid or blood contained within the pressure lumen is symbolized by Lₚ and represents mass per unit area of the fluid within the proximal pressure lumen. The resistance to fluid flow is symbolized by Rₚ and represents viscous loss as described by Poiseuille's Law, Rₚ=8 uL/Pi×R⁴, where u=viscosity, L-length of the tubing or double lumen shaft, for example, and R is the hydraulic radius of proximal lumen of the double lumen shaft, for example (i.e., ½ of the hydraulic diameter). The total compliance is symbolized by Cₜ and represents the summation of compliances due to the presence of bubbles in the proximal pressure lumen, compliance of the pressure transducer diaphragm, compliance due to the fluid, such as blood contained within the proximal pressure lumen and compliance of the double lumen shaft material. The double lumen shaft can be constructed from polyethylene, nylon, Pebax, or other materials commonly used in catheter construction for delivery of pressure signals within the body; the compliance of such material describes the change in Radius of a pressure lumen such as the proximal pressure lumen per the change in pressure contained within the pressure lumen. A double lumen shaft constructed with a material having a larger elastic modulus will have a smaller compliance.

The equation that describes flow within the proximal pressure lumen as a function of time has two components, a natural component and a forced component that depends upon the nature of the pressure signal being generated by the heart. The natural component describes the natural frequency of the system which includes the double lumen shaft, the shaft compliance, shaft length, and proximal lumen hydraulic diameter. The natural frequency of the system, fo, is described by:

fo=(½Pi)(R²−(4L/C))^{1/2}

In constructing the double lumen shaft of the present invention, one desires fo to be larger than about 40 Hz. If R²>4L/C the system is overdamped, and the transmitted signal delivered to the manifold can be of a smaller amplitude than the actual signal and higher frequency signals being generated by the heart can be missed. If R²<4LC, the system is underdamped, and the transmitted signal can have “ringing” and thereby transmitting signals that are not being generated by the heart but are associated with the natural frequency of the system. The double lumen shaft of the present invention is designed to deliver a critically damped signal along the proximal pressure lumen by setting R2=4L/C. The hydraulic diameter (i.e., two-time R) is one of the most demonstrative factors that determines the ability of the double lumen shaft of the present invention to transmit signals accurately and with fidelity to the pressure transducer located at the manifold. The hydraulic radius of the proximal pressure lumen is equal to (4L/C)^{1/2}.

The present catheter designs provides advantages to existing designs. For example, on a safety front, two lumens contained within a single extrusion cannot be separated by power injection force. The present design offers the opportunity to eliminate the risk of component embolism. Also on a safety front, a single extrusion design can reduce or eliminate dead spaces in a proximal shaft segment of the catheter, thereby simplifying flush preparation and reducing risk of air embolization. On an efficacy front, the single, relatively rigid extrusion containing independent, non-coaxial lumens can overcome the potential for either lumen to be obstructed due to relative movement of one catheter component within the other. The non-coaxial lumen design can also overcome high shear forces inherent to coaxial designs, which has implications for both signal fidelity and injection flow rates.

### ELEMENT NUMERAL REFERENCE GUIDE

In the drawings, like numerals can be used to describe similar features and components throughout the several views.

| | |
|---|---|
| 100/1300/1400/1600 | Double lumen pigtail |
| | catheter |
| 1201/1301/1401/1601 | Guidewire |
| 102/202/402/602/802/902/ | Double lumen shaft |
| 1102/1902/2002 | |
| 1203/1303/1403 | (Cardiac) diagnostic |
| | catheter |
| 104/304/404/604/804/1004/ | Single lumen shaft |
| 1104 | |
| 1205/1305 | Diagnostic catheter hub |
| 106/506/1106/1606/1706 | Proximal shaft segment |
| 107 | Proximal segment axis |
| 108/408/508/608/808/1108/ | Distal shaft segment |
| 1408/1508/1608 | |
| 109 | Distal segment axis |
| 110/210/1110/1910 | Proximal pressure lumen |
| 811/1011 | Reflowable, collapsed |
| | proximal pressure (oval) |
| | lumen |
| 112/212/312/412/712/812/ | Distal pressure lumen |
| 1012/1112/1312/1612/ | |
| 1712/1912 | |
| 913/1013 | Collapsed oval lateral wall |
| 114/1114/1314 | Manifold (with optional |
| | hub interlock) |
| 1215 | Diagnostic catheter distal |
| | opening |
| 116/1116/1316/1616 | Proximal pressure port |
| 1217/1317 | Diagnostic catheter distal |
| | segment |
| 118/1118/1318/1618 | Distal pressure port |
| 120 | Proximal orifice region |
| 122/222/422/522/622/822/ | Proximal orifice(s) |
| 1122/1322/1522/1622/1722/ | |
| 1822/1922 | |
| 223 | Proximal orifice diameter |
| 124/424/524/624/724/824/ | Distal orifice(s) |
| 1024/1124/1324/1524/ | |
| 1624/1724 | |
| 126 | Proximal direction |
| 128/228/428/628/728/828 | Braided structure |
| 130/530 | Braided pre-bend region |
| 132/432/632/832/1332 | Shaft junction |
| 134/534/834/1134/1834 | Shaft bend/shaft curve |
| 136/536/1736 | Shaft bend angle |
| 138 | Post-bend region |
| 1739 | Neighboring regions |
| 140/540/840/1740 | Shaft (braided) post-bend |
| | region |
| 1741 | Opposing wall |
| 142/542/842/1742 | Flexible linear region |
| 1743 | Coil center |
| 144/444 | Distal orifice region |
| 145/445/545/645/845/ | Coil |
| 1545/1845 | |
| 146/646 | Coil region |
| 147 | Coil apex |
| 148/1748 | Coil diameter |
| 149/1749/1849 | Radiopaque marker |
| 150/550/1650/1750 | Distal end/distal end |
| | opening |
| 451/551 | Pigtail coil plane |
| 152/2052 | Shaft bend plane |
| 1753 | Coil perimeter |
| 154/554/1254/1454/ | Aorta |
| 1554/1654 | |
| 1255 | Aortic root |
| 156 | Aortic axis |
| 158 | Sinotubular junction |
| 160/560/1260/1360/1460/ | Left ventricle |
| 1560/1660/1760/1860 | |
| 1261 | Endocardial surface |
| 162 | Left ventricular chamber |
| | axis |
| 1463 | Left ventricular central |
| | region |
| 164 | Left ventricular |
| | inferobasilar wall |
| 166/1266/1466/1566/ | Left ventricular apex |
| 1766/1866 | |
| 567/1267/1367/1467/ | Aortic valve |
| 1567/1667 | |
| 168/1768/1868 | Aortic valve cusp |
| 170/1270/1370/1470/ | Aortic leaflet(s) |
| 1570/1670 | |
| 1971 | (Circular) lateral wall fiber |
| 272/372/772/872/ | Circular lateral wall |
| 972/1072/1972 | |
| 1973 | (Oval) lateral wall fiber |
| 274/374/774/874/ | Common wall |
| 974/1074/1974 | |
| 1975 | Distal pressure lumen |
| | center |
| 276/876/1976 | Oval lateral wall |
| 477/1777/2077 | Outer surface |
| 278/378 | Distal lumen diameter |
| 479 | Inner curve surface |
| 280 | Hydraulic diameter |
| 1781 | Inner surface |
| 282/1182/2082 | Proximal pressure lumen |
| | major diameter |
| 483/1783 | In-plane surface |
| 284/1184/2084 | Proximal pressure lumen |
| | minor diameter |
| 1785/1885 | Hypertrophic cardiac |
| | muscle (proximal septal |
| | muscle) |
| 586 | Cordae tendineae |
| 1987 | Proximal pressure lumen |
| | center |
| 588 | Inferior wall |
| 589 | Septal wall |
| 590 | Lateral wall |
| 592/1792 | Mitral leaflet |
| 594/1294 | Mitral valve orifice |
| 2195 | Simplified component |
| | model |
| 896/996/1096 | Elastic member |
| 2097 | Double lumen shaft major |
| | diameter |
| 1098 | Attachment |
| 2099 | Double lumen shaft minor |
| | diameter |

The above Detailed Description includes references to the accompanying drawings, which form a part of the Detailed Description. The Detailed Description should be read with reference to the drawings. The drawings show, by way of illustration, specific embodiments in which the present catheters and related methods can be practiced. These embodiments are also referred to herein as “examples.” The use of “adapted to,” “configured to,” or similar herein is meant as open and inclusive language that does not foreclose devices or components adapted to or configured to perform additional functions. The use of “proximal” and “distal” herein refer to relative positions with respect to a user of the elongate, minimally invasive devices, where “proximal” means relatively towards the user and “distal” means relatively away from the user. Headings, lists, and numbering included herein are for ease of explanation only and are not meant to be limiting. All numeric values are assumed to be modified by the term “about,” whether or not explicitly indicated. The term “about” generally refers to a range of numbers that one of ordinary skill would consider equivalent to the recited value (e.g., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.).

The Detailed Description is intended to be illustrative and not restrictive. For example, the above-described examples (or one or more features or components thereof) can be used in combination with each other. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:

In Example 1, a pigtail catheter configured to measure a pressure proximal and distal to a narrowing can comprise a proximal shaft segment and a distal shaft segment. The proximal shaft segment can include double lumen tubing defining a proximal pressure lumen and a non-coaxial, distal pressure lumen. The distal shaft segment can be configured to be partially positioned across the narrowing and has a portion that includes the distal pressure lumen but not the proximal pressure lumen. The distal shaft segment can include at least one distal orifice positionable distal to the narrowing and having a diameter of at least about 0.018 inches, and at least one proximal orifice positionable proximal to the narrowing and having a diameter of at least about 0.018 inches.

In Example 2, the pigtail catheter of Example 1 can optionally be configured such that the distal pressure lumen has a generally circular cross-sectional shape, and the proximal pressure lumen has a generally crescent or kidney cross-sectional shape that wraps partially around the distal pressure lumen.

In Example 3, the pigtail catheter of any one of Examples 1 or 2 can optionally further comprise a manifold coupled to a proximal end of the proximal shaft segment. The manifold can include a proximal pressure port in fluid communication with the proximal pressure lumen and a distal pressure port in fluid communication with the distal pressure lumen.

In Example 4, the pigtail catheter of Example 3 can optionally be configured such that the manifold is configured to deliver a proximal pressure signal and a distal pressure signal to a transducer for determination of a pressure gradient across the narrowing.

In Example 5, the pigtail catheter of any one or any combination of Examples 1-5 is optionally configured such that the proximal shaft segment includes a braided structure contained within an outer wall of the double lumen tubing.

In Example 6, the pigtail catheter of Example 5 is optionally configured such that the braided structure comprises metallic or polymeric fibers having a spacing of at least about 0.020 inches to allow for placement of a proximal orifice therebetween.

In Example 7, the pigtail catheter of any one of Examples 5 or 6 is optionally configured such that the braided structure extends to a portion of the distal shaft segment.

In Example 8, the pigtail catheter of any one or any combination of Examples 1-7 is optionally configured such that an outer wall of the double lumen tubing comprises a first wall fiber adjacent to the proximal pressure lumen. The first wall fiber can extend in an axial direction and have a non-extensional characteristic.

In Example 9, the pigtail catheter of Example 8 is optionally configured such that the outer wall of the double lumen tubing comprises a second wall fiber adjacent to the distal pressure lumen. The second wall fiber can extend in an axial direction and have a non-extensional characteristic.

In Example 10, the pigtail catheter of any one or any combination of Examples 1-10 can optionally be configured such that an outer surface of the double lumen tubing has an oval cross-sectional shape defining a shaft major axis and a shaft minor axis, and a center of the distal pressure lumen and a center of the proximal pressure lumen are located on the shaft major axis.

In Example 11, the pigtail catheter of Example 10 can optionally be configured such that the outer surface has a shaft minor length that is at least ten percent (10%) less than a shaft major length.

In Example 12, the pigtail catheter of any one or any combination of Examples 1-11 optionally further comprises an elastic member positioned within a distal portion of the proximal pressure lumen and forming an attachment to the distal shaft segment.

In Example 13, the pigtail catheter of Example 12 is optionally configured such that the elastic member extends along a length of the distal shaft segment, including a pigtail coil at the end of the distal shaft segment.

In Example 14, the pigtail catheter of any one or any combination of Examples 1-13 is optionally configured such that the distal shaft segment includes a shaft bend distal to the at least one proximal orifice.

In Example 15, the pigtail catheter of Example 14 is optionally configured such that the shaft bend forms a shaft bend angle ranging from about 145 degrees to about 165 degrees, inclusive.

In Example 16, the pigtail catheter of any one or any combination of Examples 1-15 is optionally configured such that the distal shaft segment includes a pigtail coil having a diameter less than or equal to about 1.5 cm.

In Example 17, the pigtail catheter of Example 16 is optionally configured such that a plane of the pigtail coil is non-coplanar with a plane of a shaft bend located in the distal shaft segment, distal to the at least one proximal orifice.

In Example 18, the pigtail catheter of Example 17 is optionally configured such that the plane of the pigtail coil is angled about 5 degrees to about 45 degrees, inclusive, relative to the plane of the shaft bend.

In Example 19, the pigtail catheter of any one or any combination of Examples 16-18 is optionally configured such that the pigtail coil includes a coil apex at a distal-most coil portion. The distal-most coil portion can have a radius of curvature that is less than a radius of curvature of the remaining portions of the pigtail coil.

In Example 20, the pigtail catheter of any one or any combination of Examples 16-19 is optionally configured such that the pigtail coil includes a coil apex at a distal-most coil portion. A radiopaque marker can be positioned at the coil apex.

In Example 21, the pigtail catheter of any one or any combination of Examples 1-20 is optionally configured such that one or both of the proximal pressure lumen and the distal pressure lumen has a hydraulic diameter of about least about 0.018 inches.

In Example 22, a method can comprise inserting a pigtail catheter into a heart such that a distal shaft segment of the catheter is partially positioned in a left ventricle and determining a pressure gradient across an aortic valve. A proximal shaft segment of the catheter can include double lumen tubing defining a distal pressure lumen having a generally circular cross-sectional shape and a proximal pressure lumen having a generally crescent or kidney cross-sectional shape that wraps partially around the distal pressure lumen. The distal shaft segment can include a portion that includes the distal pressure lumen but not the proximal pressure lumen, at least one proximal orifice positioned proximal to an aortic valve, and at least one distal orifice positioned distal to the aortic valve. Determining the pressure gradient across the aortic valve can include coupling a pressure transducer to a manifold of the pigtail catheter. The manifold can include a proximal pressure port in communication with the proximal pressure lumen and a distal pressure port in communication with the distal pressure lumen.

In Example 23, the method of Example 22 can optionally be configured such that inserting the pigtail catheter into the heart includes inserting the pigtail catheter over a cardiac diagnostic catheter using the distal pressure lumen.

In Example 24, the method of Example 23 can optionally further comprise removing the cardiac diagnostic catheter from the distal pressure lumen.

In Example 25, the method of any one of Examples 22-24 can optionally be configured such that one or both of the proximal pressure lumen and the distal pressure lumen include a hydraulic diameter of at least about 0.018 inches.

The scope of the present catheter and related methods should be determined with reference to the appended claims, along with the full scope of equivalents to which such claims are entitled. In the appended claims, the terms “including” and “in which” are used as the plain-English equivalents of the respective terms “comprising” and “wherein.” Also in the following claims, the terms “including” and “comprising” are open-ended; that is, a catheter or method that includes features, components, or steps in addition to those listed after such a term in a claim are still deemed to fall within the scope of that claim. Moreover, the terms “first,” “second,” “third,” etc. in the following claims are used merely as labels, and such terms not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A pigtail catheter configured to measure a pressure proximal and distal to a narrowing, comprising:
a proximal shaft segment including double lumen tubing defining a proximal pressure lumen and a non-coaxial, distal pressure lumen; and
a distal shaft segment configured to be partially positioned across the narrowing and having a portion that includes the distal pressure lumen but not the proximal pressure lumen,
the distal shaft segment including at least one distal orifice positionable distal to the narrowing and having a diameter of at least about 0.018 inches, and at least one proximal orifice positionable proximal to the narrowing and having a diameter of at least about 0.018 inches.

2. The pigtail catheter of claim 1, wherein the distal pressure lumen has a generally circular cross-sectional shape, and wherein the proximal pressure lumen has a generally crescent or kidney cross-sectional shape that wraps partially around the distal pressure lumen.

3. The pigtail catheter of claim 1, further comprising a manifold coupled to a proximal end of the proximal shaft segment, the manifold including a proximal pressure port in fluid communication with the proximal pressure lumen and a distal pressure port in fluid communication with the distal pressure lumen.

4. The pigtail catheter of claim 3, wherein the manifold is configured to deliver a proximal pressure signal and a distal pressure signal to a transducer for determination of a pressure gradient across the narrowing.

5. The pigtail catheter of claim 1, wherein the proximal shaft segment includes a braided structure contained within an outer wall of the double lumen tubing.

6. The pigtail catheter of claim 5, wherein the braided structure extends to a portion of the distal shaft segment.

7. The pigtail catheter of claim 1, wherein an outer wall of the double lumen tubing comprises a first wall fiber adjacent to the proximal pressure lumen, the first wall fiber extending in an axial direction and having a non-extensional characteristic.

8. The pigtail catheter of claim 7, wherein the outer wall of the double lumen tubing comprises a second wall fiber adjacent to the distal pressure lumen, the second wall fiber extending in an axial direction and having a non-extensional characteristic.

9. The pigtail catheter of claim 1, wherein an outer surface of the double lumen tubing has an oval cross-sectional shape defining a shaft major axis and a shaft minor axis, and wherein a center of the distal pressure lumen and a center of the proximal pressure lumen are located on the shaft major axis.

10. The pigtail catheter of claim 1, further comprising an elastic member positioned within a distal portion of the proximal pressure lumen and forming an attachment to the distal shaft segment.

11. The pigtail catheter of claim 10, wherein the elastic member extends along a length of the distal shaft segment, including a pigtail coil at the end of the distal shaft segment.

12. The pigtail catheter of claim 1, wherein the distal shaft segment includes a shaft bend distal to the at least one proximal orifice.

13. The pigtail catheter of claim 12, wherein the shaft bend forms a shaft bend angle ranging from about 145 degrees to about 165 degrees, inclusive.

14. The pigtail catheter of claim 1, wherein the distal shaft segment includes a pigtail coil having a diameter less than or equal to about 1.5 cm.

15. The pigtail catheter of claim 14, wherein a plane of the pigtail coil is non-coplanar with a plane of a shaft bend located in the distal shaft segment, distal to the at least one proximal orifice.

16. The pigtail catheter of claim 1, wherein one or both of the proximal pressure lumen and the distal pressure lumen has a hydraulic diameter of about least about 0.018 inches.

17. A method, comprising:
inserting a pigtail catheter into a heart such that a distal shaft segment of the catheter is partially positioned in a left ventricle, wherein a proximal shaft segment of the catheter includes double lumen tubing defining a distal pressure lumen having a generally circular cross-sectional shape and a proximal pressure lumen having a generally crescent or kidney cross-sectional shape that wraps partially around the distal pressure lumen, and wherein the distal shaft segment includes a portion that includes the distal pressure lumen but not the proximal pressure lumen, at least one proximal orifice positioned proximal to an aortic valve, and at least one distal orifice positioned distal to the aortic valve;
determining a pressure gradient across the aortic valve, including coupling a pressure transducer to a manifold of the pigtail catheter, the manifold including a proximal pressure port in communication with the proximal pressure lumen and a distal pressure port in communication with the distal pressure lumen.

18. The method of claim 17, wherein inserting the pigtail catheter into the heart includes inserting the pigtail catheter over a cardiac diagnostic catheter using the distal pressure lumen.

19. The method of claim 18, further comprising removing the cardiac diagnostic catheter from the distal pressure lumen.

20. The method of claim 17, wherein one or both of the proximal pressure lumen and the distal pressure lumen include a hydraulic diameter of at least about 0.018 inches.
